(19) United States
(12) Patent Application Publication
(10) Pub. No.: US 2004/0180403 A1
(43) Pub. Date: Sep. 16, 2004

(54) Novel 33 phage vectors

(76) Inventors: Katherine S. Bowdish, Del Mar, CA (US); Shana Barbas-Frederickson, Solana Beach, CA (US)

Correspondence Address: Mark Farber, Alexion Pharmaceuticals Inc, 352 Knotter Drive, Cheshire, CT, 06410, US

(21) Appl. No.: 10/475,463

(22) PCT Filed: Apr. 26, 2002
(86) PCT No.: PCT/US02/13610

(51) Int. Cl. C12N 5/06; C12N 15/86

(52) U.S. Cl. 435/69.1; 435/456; 435/326

(57) ABSTRACT

A phage genome is engineered to include a novel restriction site at one of two different positions. In a first embodiment, a restriction site is inserted into the phage genome I between the end of gene IV and the MOS hairpin which serves as a phage packaging signal for newly synthesized single strands of phage DNA. In a second embodiment, a restriction site is inserted into the phage genome after the MOS hairpin and prior to the minus strand origin. Once the phage genome is modified to contain the new restriction site, the vector can be engineered to be a “33” vector by inserting at the new restriction site a nucleotide sequence encoding at least a functional domain of pIII and at least a first cloning site for receiving a gene encoding a polypeptide to be displayed and, optionally a second cloning site for receiving a second gene encoding a polypeptide capable of dimerizing with the polypeptide to be displayed. In particularly useful embodiments, the novel vectors are engineered to produce phage particles that display antibodies.

### BACKGROUND

1. Technical Field

This disclosure relates to phage vectors useful for generating phage display libraries. More specifically this disclosure relates to vectors useful for display of antibodies on phage particles.

2. Background of Related Art

Filamentous bacteriophage consist of a circular, single-stranded DNA molecule surrounded by a cylinder of coat proteins. There are about 2,700 molecules of the major coat protein pVIII that envelope the phage. At one end of the phage particle, there are five copies each of gene III and VI proteins (pIII and pVI) that are involved in host-cell binding and in the termination of the assembly process. The other end contains five copies each of pVII and pIX that are required for the initiation of assembly and for maintenance of virion stability.

In recent years, vectors have been developed that allow the display of foreign peptides on the surface of a filamentous phage particle. By insertion of specific oligonucleotides or entire protein coding regions into genes encoding specific phage capsid proteins, chimeric proteins can be produced which are able to be assembled into phage particles. This results in the display of the foreign protein or peptide on the surface of the phage particle.

The display of peptides and proteins on the surface of bacteriophage represents a powerful methodology for selection of rare members in a complex library and for carrying out molecular evolution in the laboratory. The ability to construct libraries of enormous molecular diversity and to select for molecules with predetermined properties has made this technology applicable to a wide range of problems.

A few of the many applications of such technology are: i) phage display of natural peptides including, mapping epitopes of monoclonal and polyclonal antibodies and generating immunogens; ii) phage display of random peptides, including mapping epitopes of monoclonal and polyclonal antibodies, identifying peptide ligands, and mapping substrate sites for proteases and kinases; and iii) phage display of protein and protein domains, including directed evolution of proteins, isolation of antibodies, and cDNA expression screening.

One important application of phage display has been to construct combinatorial peptide libraries. Synthetic oligonucleotides, fixed in length but with unspecified codons, can be cloned as fusions to genes III or VIII of phage where they are expressed as a plurality of peptide:capsid fusion proteins. The libraries, often referred to as random peptide libraries, can then be tested for binding to target molecules of interest This is most often done using a form of affinity selection known as “biopanning” or simply “panning”.

A variety of commonly used display vectors, with their name, site of expression, restriction site used and marker carried on the vector, are provided in Phage Display of Peptides and Proteins, A Laboratory Manual, ed. Kay et al., Academic Press, 1996, page 38 and reproduced in the following table:

| Vector | Gene | Rest. Site(s) | Marker |
|---|---|---|---|
| fUSE5 | III | BglI-S-BglI | tet^{R} |
| fAFF1 | III | BstXI-S-BstXI | tet^{R} |
| fd-CAT1 | III | PstI-S-XhoI | tet^{R} |
| M663 | III | XhoI-S-XbaI | lacZ⁺ |
| fdtetDOG | III | ApaLI-S-NotI | tetR |
| 33 | III | SfiI-S-NotI | |
| 88 | VIII | | |
| Phagemid | III | | amp^{R} |
| pHEN1 | III | SfiI-S-NotI | amp^{R} |
| pComb3 | III | | |
| pComb8 | VIII | | |
| pCANTAB 5E | III | SfiI-SNotI | amp^{R} |
| p8V5 | VIII | BstXI-S-BstXI | amp^{R} |
| λSurfZap | III | NotI-S-SpeI | amp^{R} |

A variety of phage and phagemid vectors have been constructed and utilized for phage display. Each of the existing vectors has its advantages and disadvantages. By convention, vectors that fuse a gene of interest whose protein product is to be displayed to gene III have been categorized as either type 3, type 3+3 or type 33. Type 3 vectors are phage vectors where all copies of gene III are fused to a gene of interest for display. M663 is an example of a phage vector where the fusion is at the native gene III site. In M663 and other type 3 vectors, all 3-5 copies of pIII on the phage surface will be fusion proteins. However, since pIII is required for attachment and infectivity of the phage particle, some proteolysis of the displayed protein is required in order to regain infectivity when large proteins are displayed.

The 3+3 vectors are phagemid vectors. In the phagemid system, helper phage are required to package the phagemid genome into a phagemid particle that is extruded out of the cell. In 3+3 vectors, the gene of interest is fused to a copy of gene III on the plasmid, while the helper phage retains a wildtype, unfused copy of gene III. Hence, the minor coat proteins of the phagemid particle are made up of both wildtype and fusion proteins leading to retention of infectivity. However, since both helper phage and phagemid particles are produced from the same cell, both helper phage and phagemid viral particles will have fusion proteins on the surface leading to a loss of the corresponding genetic information from helper phage particles that inadvertently display selected fusion proteins.

The type 33 vectors are phage vectors where both a fused and unfused copy of gene III are present on the phage vector. The phage vector system is less complex in that helper phage are not required. Additionally, there is no loss of selected clones that result from inadvertent display on the helper phage surface. However, most presently known phage vectors are a derivative of fd-tet where an insert conferring tetracycline resistance was introduced at a convenient restriction site. Unfortunately, the insert disrupts the minus strand origin of replication, leading to a defect in minus strand synthesis. As a result, these vectors have a very low intracellular RF copy number, making vector production for cloning as well as library amplification difficult. In addition, the size of the insert conferring tetracycline resistance is approximately 2.6 kb. This large insert, in addition to insertions into the phage for protein of interest display (including promoter, ribosomal binding sites, signal sequences, stuffer fragments in the case of the cloning vectors, and antibody genes in the case of antibody display) yield a large phage genome that is not packaged as efficiently as smaller phage genomes. The fd-tet vector has served as the starting point of construction of a variety of phage vectors including the fUSE vectors (Scott and Smith, Science, Vol. 249, pages 386-390, 1990), fd-CAT1 (McCafferty et al., Nature (London), Vol. 348, pages 552-554, 1990) and fdtetDOG (Hoogenboom et al., Nucleic Acid Res., Vol. 19, pages 4133-4137, 1991).

### SUMMARY

This disclosure describes novel phage vectors useful for generating phage display libraries. The novel vectors described herein are produced as the result of modification of a phage genome at an artificially created cloning site not employed in previous phage vector constructions.

Specifically, a phage genome is engineered in accordance with this disclosure to include a restriction site at one of two different positions. In a first embodiment, a restriction site is inserted into the phage genome between the end of gene IV and the MOS hairpin which serves as a phage packaging signal for newly synthesized single strands of phage DNA. In a second embodiment, a restriction site is inserted into the phage genome after the MOS hairpin and prior to the minus strand origin.

Once the phage genome is modified to contain the new restriction site, cloning sites for receiving one or more genes can be inserted into the phage vector in accordance with this disclosure. Preferably, the vector is engineered to be a “33” vector by inserting at the new restriction site a nucleotide sequence encoding at least a functional domain of pIII and at least a first cloning site for receiving a gene encoding a polypeptide to be displayed. In an alternative embodiment, the 33 vector is engineered to cause display of a dimeric (e.g., heterodimeric) species by inserting first cloning site for receiving first gene encoding a polypeptide to be displayed and a second cloning site for receiving a second gene encoding a polypeptide capable of dimerizing with the polypeptide to be displayed, thereby resulting in display of a dimeric polypeptide or protein. The first and second cloning sites, if desired and practical, can be inserted with the nucleotide sequence encoding at least a functional domain of pIII as part of a single cassette referred to herein as a display cassette.

In particularly useful embodiments, the novel vectors are engineered to produce phage particles that display antibodies. After creation of the novel restriction site and insertion of the display cassette within a phage genome, a first gene encoding an antibody heavy chain Fd is inserted at a first cloning site adjacent the nucleotide sequence encoding at least a functional domain of pIII to produce a pIII fused with a heavy chain Fd. A second gene encoding an antibody light chain is also inserted into the vector at a second cloning site.

### BRIEF DESCRIPTION OF THE DRAWINGS

FIG. 1 is a flow chart illustrating the strategy for making a vector based on modification of the f1 genome between gene IV and the MOS hair pin;

FIG. 2 is a flow chart illustrating the strategy for making a vector based on modification of the f1 genome between the MOS hairpin and the minus strand origin;

FIG. 3 is a map of the vector produced in Example 1;

FIG. 4a is the sequence (Seq. ID No. 2) of cassette 1a used in Example 1;

FIG. 4b is the sequence (Seq. ID No. 7) of cassette 2 used in Examples 1 and 2;

FIG. 4c is the sequence (Seq. ID No. 12) of cassette 3 used in Examples 1 and 2;

FIG. 5 is a map of vector pXA131;

FIGS. 6a-d show the nucleotide sequence (Seq. ID No. 13) of vector pAX131;

FIGS. 7a, 7b and 7c show the sequences of overlapping oligos for producing fragments A, B and C, respectively of the display cassette in Example 1;

FIGS. 8, 9a-b and 10 show the alignment of the oligos for fragments A, B and C, respectively;

FIG. 11 is a map of the vector pAX131-syn.gIII;

FIGS. 12a-b show the sequence (Seq. ID No. 64) of the final construct resulting from the insertion of cassettes 1a, 2 and 3 in Example 1;

FIG. 13 is a map of the vector produced in Example 2;

FIG. 14 is the sequence (Seq. ID No. 66) for cassette 1b used in Example 2; and

FIGS. 15a-c is the sequence (Seq. ID No. 71) of the final construct resulting from the insertion of cassettes 1b, 2 and 3 as described in Example 2.

### DETAILED DESCRIPTION OF PREFERRED EMBODIMENTS

The novel vectors described herein are prepared by modifying a phage genome. While the following description is provided with respect to the f1 genome as the starting material, it should be understood that other phage genomes (e.g., M13, fd, etc.) can be used as the starting material. Additionally, when the following description refers to “pIII” it should be understood that either full pIII or a truncated version or fragment thereof is contemplated (unless the context indicates otherwise) provided the display function is maintained.

In one embodiment, the present vectors are the result of modification of the f1 genome between gene IV and the hairpin which serves as a packaging signal (MOS). First, the phage genome is engineered to contain a novel restriction site at this location. Then at least a first cloning site and a nucleotide sequence encoding at least a functional domain of pIII are inserted at the newly formed restriction site. A first gene encoding a polypeptide to be displayed can be inserted at the first cloning site. Because the first cloning site is adjacent the nucleotide sequence encoding at least a functional domain of pIII, once the first gene is inserted, the vector effectively encodes a fusion protein of pIII and a polypeptide to be displayed by the phage particle. Any polypeptide that can be displayed by phage can be fused to pIII. Non-limiting examples of polypeptides that can be displayed include naturally occurring and synthetic enzymes, hormones, antibodies, antigens, toxins and cytokines. For a nonlimiting list of proteins and protein domains that can be displayed, see Phage Display of Peptides and Proteins, A Laboratory Manual, Kay et al., ed., Academic Press, 1996.

Optionally, a second cloning site is also inserted at the novel restriction site. The second cloning site is adapted to receive a second gene that encodes a polypeptide that can dimerize with the polypeptide fused to pIII. In this manner, display of a dimeric species (e.g., a heterodimeric species) can be achieved. Where monomeric display of a single polypeptide or protein is intended, the second gene can be eliminated.

In particularly preferred embodiments, the vector can be used to make phage particles that display antibody libraries. Unless the context indicates otherwise, the term “antibody” as used herein refers to any molecule that contains at least one immunologically active portion, and includes whole immunoglobulin molecules, single chain antibodies, Fab, F(ab′)2, scFv, Fv, heavy chain variable regions and light chain variable regions. Thus, in one useful embodiment, the polypeptide fused to pIII is an antibody heavy chain Fd and the modification to the f1 genome also involves inserting a site for cloning into the vector a second gene encoding an antibody light chain.

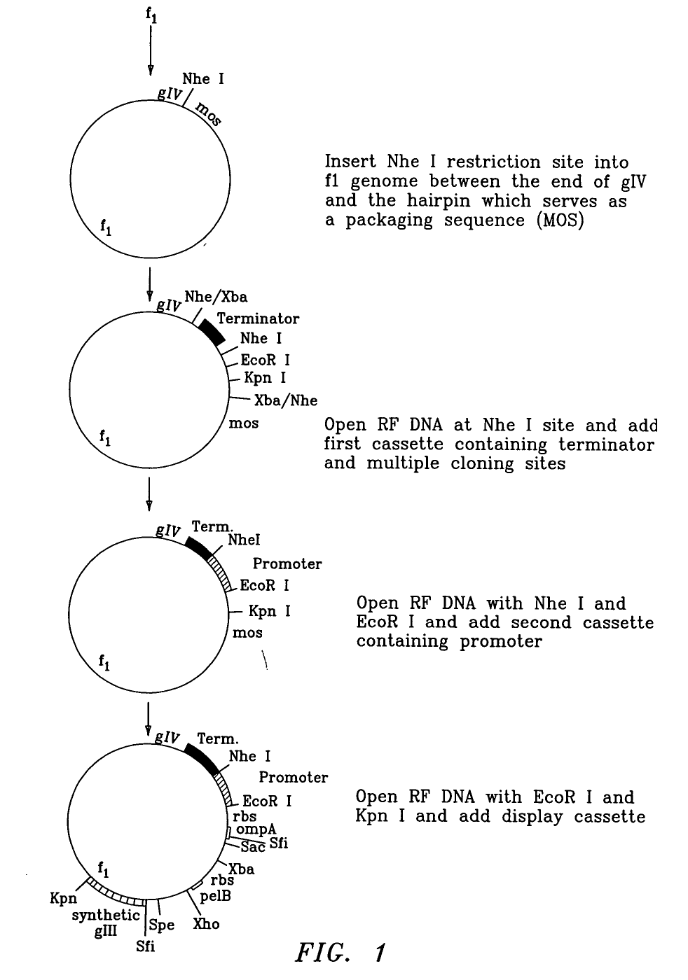

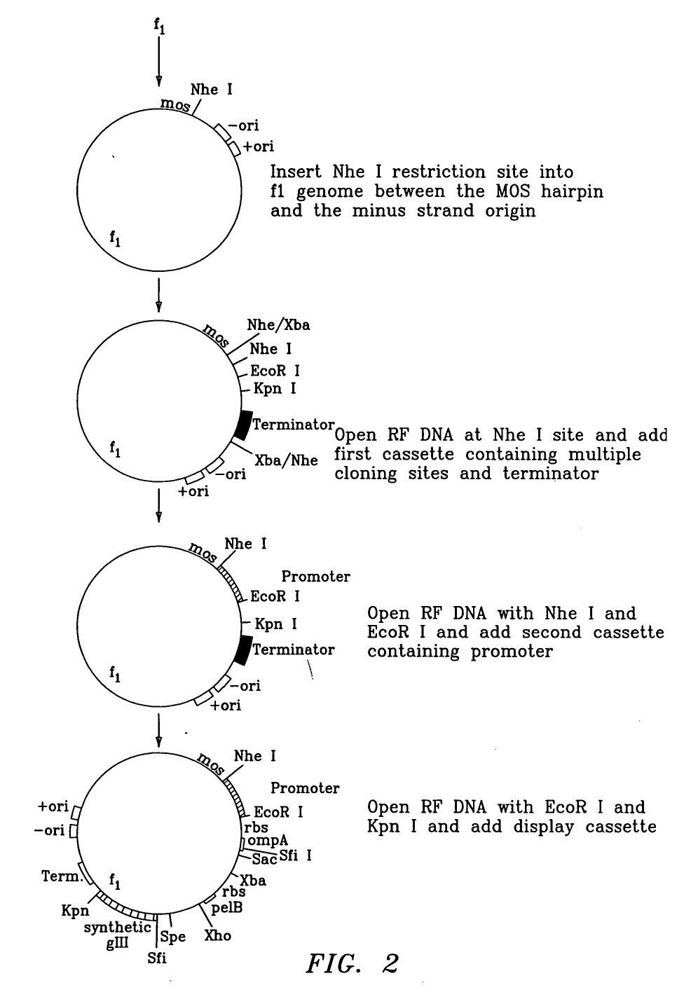

FIG. 1 is a flow chart showing the steps involved in a particularly useful method for producing a phage vector capable of generating phage display of polypeptides (e.g., libraries of antibodies) in accordance with this disclosure. In the first step, a restriction site is introduced into the into the f1 genome between the end of gene IV and the hairpin which serves as a packaging signal (MOS). The restriction site can be any known restriction site. Suitable restriction sites for insertion include, but are not limited to Nhe I, Hind III, Nco I, Xma I, Bgl II, Bst I, Pvu I, etc. It should be understood that if a restriction site selected for insertion is present in the native genome, it may be desirable to remove or disable the native restriction site to avoid unwanted digestion during further processing. The restriction site can be inserted using any technique known to those skilled in the art. In a particularly useful embodiment, site specific mutagenesis is used to incorporate the restriction site using single stranded DNA (+strand) as a template.

In the next step, the replicative form (RF) DNA is opened by digestion and a first cassette containing a terminator and multiple cloning sites is added. Depending on the particular restriction site inserted in the first step, specific methods for opening the RF DNA are known to and readily selected by those skilled in the art Preferably the first cassette is engineered to include overhangs which, when combined with the ends of the DNA formed by the digestive opening thereof at the inserted restriction site will create a hybrid site that will no longer be recognized as the inserted restriction site. In this manner, subsequent cloning steps advantageously occur at the cloning sites within the first cassette. If desired, one of the cloning sites within the first cassette can be the same as the restriction site inserted in the first step to decrease the number of different enzymes employed in the process.

Methods of preparing suitable cassettes for this and subsequent steps are within the purview of those skilled in the art. For example, suitable cassettes can be created using overlapping oligonucleotides (“oligos”) in a PCR fill in reaction. As another example, cassettes can be created using long complementary oligios which can form a double stranded DNA cassette. The oligos are mixed in a 1:1 ratio, heat denatured and slowly cooled to allow the duplexed cassette to form. Other suitable techniques for creating cassettes will be evident to those skilled in the art.

Next, the process shown in FIG. 1 involves again opening the RF DNA at one of the cloning sites within the first cassette and inserting a second cassette that includes a promoter. Any promoter recognized by a host cell can be employed. Suitable promoters include, but are not limited to, ara, lac and trc promoters. The promoter drives expression of other sequences inserted into the vector, such as, for example expression of the pIII fusion protein and any polypeptides intended to dimerize therewith.

After insertion of the second cassette, the RF DNA is again opened at one of the other cloning sites contained in the first cassette, and a display cassette is added. As noted above, the display cassette contains at least a nucleotide sequence encoding at least a functional domain of pIII and a first cloning site adapted to receive a gene encoding a polypeptide to be displayed. The nucleotide sequence encoding at least a functional domain of pIII can be natural or synthetic. Preferably, the display cassette contains a synthetic gene III to avoid having identical native gene III sequences at two different locations within the vector. The nucleotide sequence can encode a truncated pIII provided the display function of the protein is maintained.

The display cassette contains at least a first cloning site for receiving a first gene encoding a polypeptide to be displayed. The cloning site is a region of the nucleic acid between two restriction sites, typically with a nonessential region of nucleotide sequence (commonly referred to as a “stuffer” sequence) positioned therebetween. In the flow chart of FIG. 1, the first cloning site is defined by XhoI and SpeI restriction sites adjacent to the synthetic gene III. As those skilled in the art will appreciate, a suppressible stop codon could be positioned between the first gene and the nucleotide sequence encoding at least a functional domain of pIII such that fusion display is obtained in a suppressing host (as long as the first gene is inserted in-frame) and a secreted protein without pIII is obtained in a non-suppressing host.

The display cassette optionally also contains a second cloning region for receiving a second gene encoding a polypeptide that can dimerize with the polypeptide to be displayed. For example, where the vector expresses a heavy chain Fd fused to pIII, the second gene preferably encodes an antibody light chain. As with the first cloning site, the second cloning site is a region of the vector between two restriction sites, typically with a stuffer positioned therebetween. In the flow chart of FIG. 1, the second cloning site is defined by SacI and XbaI restriction sites. It should of course be understood that where a polypeptide other than an antibody is to be displayed (such as, for example, where monomeric display of a single polypeptide or protein is intended) a second gene need not be cloned into the vector. In such cases the second cloning site can either remain unused, or be eliminated entirely. As those skilled in the art will also appreciate, where a single chain antibody is encoded by the first gene, there is no need to insert a second gene into the vector at the second cloning site.

Thus, the phage vector produced by the process illustrated in FIG. 1 will be a modified f1 genome that contains, after the native gene IV but before the MOS hairpin, a terminator, a promoter, a cloning region for receiving a gene encoding an antibody light chain, a cloning region for receiving a gene encoding an antibody heavy chain Fd to be displayed and a synthetic gene III.

In another embodiment, the present vectors are the result of modification of the f1 genome between the hairpin which serves as a packaging signal (MOS) and the minus strand origin. After engineering a novel restriction site at this location, the vector has inserted at this site at least a nucleotide sequence encoding a pIII and a cloning site for receiving a first gene encoding a polypeptide to be fused to pIII and thus displayed by the phage particle. Suitable polypeptides to be displayed are those described above in connection with the previous embodiment. Optionally, a cloning site for receiving a second gene is also inserted at this site. The second gene preferably encodes a polypeptide that can dimerize with the polypeptide fused to pIII. In this manner, display of a dimeric species (e.g., a heterodimeric species) can be achieved. Where monomeric display of a single polypeptide or protein is intended, the second gene can be eliminated.

In a particularly preferred embodiment, the polypeptide fused to pIII is a heavy chain Fd and the modification to the f1 genome also involves inserting a site for cloning into the vector a second gene encoding an antibody light chain. In this manner, the vector can be used to make phage particles that display antibody libraries.

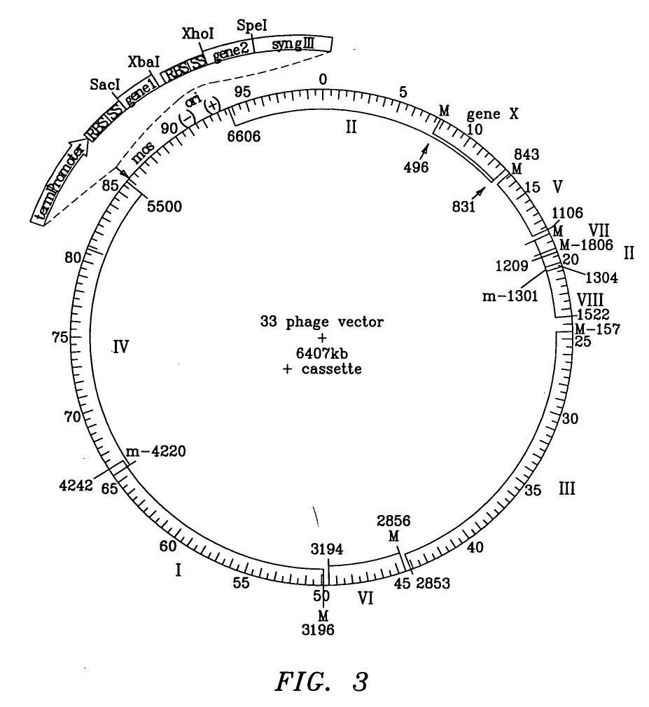

An example of a method of this alternative embodiment of forming a phage vector for generating phage display libraries of antibodies in accordance with this disclosure is shown in the flow chart of FIG. 2. In this embodiment, the first step involves introducing a restriction site into the f1 genome between the hairpin which serves as a packaging signal (MOS) and the minus strand origin. The restriction site can be any known restriction site. Suitable restriction sites for insertion include Nhe I, Hind III, Nco I, Xma I, Bgl II, Bst I, Pvu I, etc. It should be understood that if a restriction site selected for insertion is present in the native genome, it may be desirable to remove or disable the native restriction site to avoid unwanted digestion during further processing. The restriction site can be inserted using any technique known to those skilled in the art. In a particularly useful embodiment, site specific mutagenesis is used to incorporate the restriction site using single stranded DNA (+strand) as a template.

In the next step, the replicative form (RF) DNA is opened by digestion and a first cassette containing multiple cloning sites and a terminator is added. Depending on the particular restriction site inserted in the first step, specific methods for opening the RF DNA will be known to and readily selected by those skilled in the art. Preferably the first cassette is engineered to include overhangs which, when ligated with the ends of the DNA formed by digestion at the inserted restriction site will create a hybrid site that will no longer be recognized as the inserted restriction site. In this manner, subsequent cloning steps advantageously occur at the cloning sites within the first cassette. If desired, one of the cloning sites within the first cassette can be the same as the restriction site inserted in the first step to decrease the number of different enzymes employed in the process.

Next, the process shown in FIG. 2 involves again opening the RF DNA at one of the cloning sites within the first cassette and inserting a second cassette that includes a promoter. Any promoter recognized by the host cell can be employed. Suitable promoters include, but are not limited to, ara, lac and trc promoters.

After insertion of the second cassette, the RF DNA is again opened at one of the other cloning sites contained in the first cassette, and a display cassette is added. As shown in the flow chart of FIG. 2, the display cassette contains a synthetic gIII and at least a first cloning site for receiving a first gene that encodes a polypeptide to be displayed, such as, for example, an antibody heavy chain Fd. The display cassette optionally contains a second cloning region for receiving a second gene, such as, for example, a gene encoding an antibody light chain. It should of course be understood that where a polypeptide other than an antibody is to be displayed (such as, for example, where monomeric display of a single polypeptide or protein or display of a single chain antibody is intended) a second gene need not be cloned into the vector.

Thus, the phage vector produced by the process illustrated in FIG. 2 will be a modified f1 genome that contains, after the MOS hairpin but before the minus strand origin, a promoter, a cloning region for receiving a gene encoding an antibody light chain, a cloning region for receiving a gene encoding an antibody heavy chain Fd to be displayed, a synthetic gene III and a terminator.

Optionally, a selectable marker can be added to the present vectors. Non-limiting examples of suitable markers include tetracycline or kanamycin resistance. There are multiple positions within the phage genome where a selectable marker could be inserted provided that transcriptional control elements are recreated if necessary so that phage particles can still be produced.

The vectors described herein can be transformed into a host cell using known techniques (e.g., electroporation) and amplified. The vectors described herein can also be digested and have a first gene and optionally a second gene ligated therein in accordance with this disclosure. The vector so engineered can be transformed into a host cell using known techniques and amplified or to effect expression of polypeptides and/or proteins encoded thereby to produce phage particles displaying single polypeptides or dimeric species. Those skilled in the art will readily envision other uses for the novel vectors described herein.

The following examples illustrate the present invention without limiting its scope.

### EXAMPLE I

A novel vector is prepared by using the phage f1 genome as the starting material. A unique Nhe I restriction site is introduced into the f1 genome (GenBank accession #NC_{—}001397) between the end of gene IV and the MOS hairpin. (See FIG. 3). Single stranded phage DNA is used as the template with the mutagenic primer 5′ CGC GCT TAA TGC GCC GCT AGC TAC AGG GCG CGT A 3′ (Seq. ID No. 1). This causes a new Nhe I site to be created, which is underlined in the primer sequence. The double underline indicates where the mismatch will occur when the primer anneals to the remaining compatible template sequence (+strand template). Mutagenesis is performed using the MutaGene® Phagemid In Vitro Mutagenesis kit (BioRad Laboratories, Hercules, Calif.). The incorporation of the new restriction site can be verified using the resulting replicative form (RF) DNA by Nhe I digestion and/or sequence analysis. Additionally, the impact on phage assembly can be determined by looking at plaque sizes of the wild type versus modified phage. Plaque assays are performed by allowing dilutions of phage to infect a bacterial host, then plating out the mixture in top agar onto an LB-agar plate. The plates are incubated overnight to allow a bacterial lawn to form. Circular areas of slower bacterial growth, which are the result of phage infection, can be easily visualized on the plate. If the site of insertion/modification of the f1 genome interferes with the phage morphogenesis cycle, then the size of the clear circular plaque for the wild type f1 will be bigger and often less turbid than that of the modified phage.

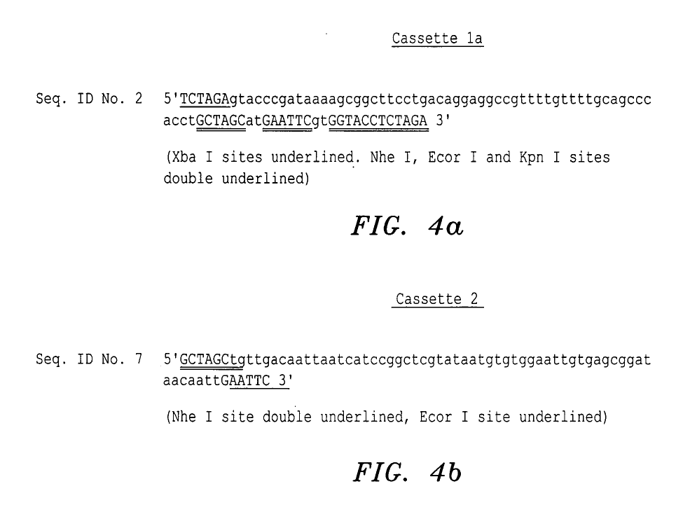

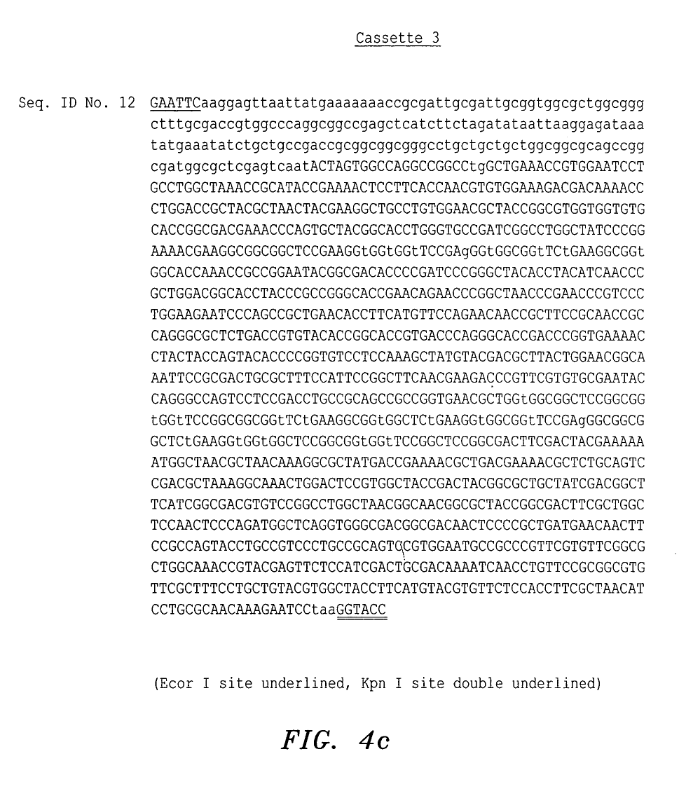

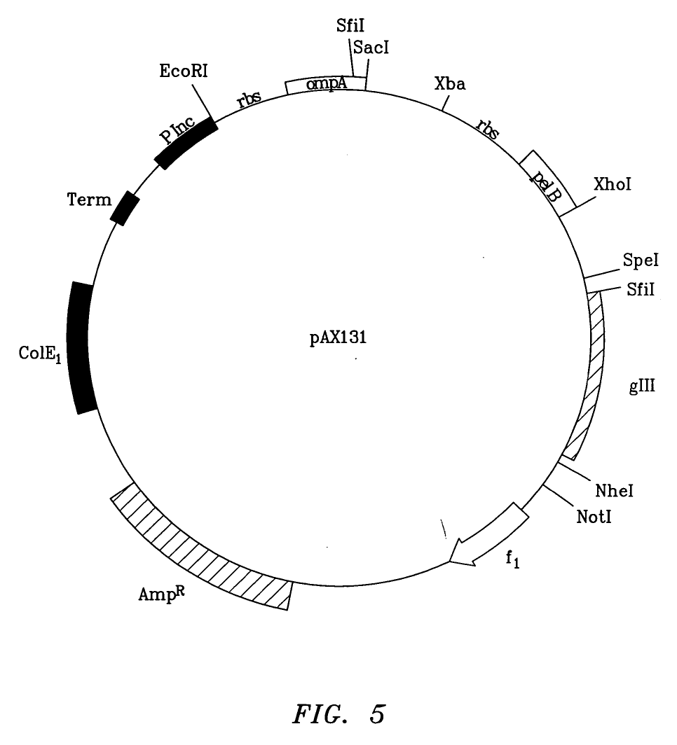

The modified f1 is digested with the restriction endonuclease Nhe I and cassette 1a (Seq. ID No. 2) (see FIG. 4a), which contains a terminator (Krebber, A., Burmester, J., and Pluckthun, A., Gene (1996) 178, pp71-4) and multiple cloning sites, is ligated into that position. Cassette 1a is created using overlapping oligos in a PCR fill in reaction. The following Oligos are used: Cas. 1a-F: 5′ gag gtt TCT AGA gta ccc gat aaa agc ggc ttc ctg aca gga ggc cgt ttt gtt ttg cag c 3′ (Seq. ID No. 3); and Cas.1a-B: 5′ aag cgt TCT AGA GGT ACC ac GAA TTC at GCT AGC agg tgg gct gca aaa caa aac ggc ctc 3′ (Seq. ID No. 4) (Xba I sites underlined; Kpn I, EcoR I and Nhe I sites double underlined). The cassette has Xba I overhangs which are compatible with Nhe I overhangs in the vector. However, on ligation the hybrid site created is not recognized as either Nhe I or Xba I. As described above, the constructs can be verified both by analysis of the RF DNA and by analyzing plaque size.

An alternative method for creating Cassette 1a makes use of long complementary oligos which can form the double stranded DNA cassette. The two oligos are mixed together at a 1:1 molar ratio, heat denatured and slowly cooled to allow the duplexed insert to form. The annealing of the oligos is such that single stranded DNA overhangs will be at each end, such as would be created after an Xba I restriction digest. Suitable oligos for use in this alternative method include:

| | |
|---|---|
| Cas. 1a-F2 | |
| 5′ CT AGA gta ccc gat aaa agc ggc | (Seq. ID No. 5) |
| ttc ctg aca gga ggc cgt ttt gtt | |
| ttg cag ccc acc t GCT AGC at | |
| GAA TTC gt GGTACC T 3′ | |
| Cas. 1a-B2 | |
| 5′ CTAGA GGTACC ac GAATTC at | (Seq. ID No. 6) |
| GCTAGC agg tgg gct gca aaa caa | |
| aac ggc ctc ctg tca gga agc cgc | |
| ttt tat cgg gta cT 3′ | |

The RF DNA is digested with Nhe I and EcoR I and cassette 2, (Seq. ID No. 7, see FIG. 4b) which contains a promoter, is added (see for example Invitrogen's pTrcHis A promoter sequence). Cassette 2 is generated using overlapping oligos in a PCR fill in reaction. The primers used are: cas.2-F 5′ ACACG CTA GCT GTT GAC AAT TAA TCA TCC GGC TCG TAT AAT GTG TGG AA 3′(Seq. ID No. 8); and cas.2-B 5′ GTCA GAA TTC AAT TGT TAT CCG CTC ACA ATT CCA CAC ATT ATA CGA GCC 3′. (Seq. ID No. 9).

An alternative method for creating Cassette 2 makes use of long complementary oligos which can form the double stranded DNA cassette. The two oligos would be mixed together at a 1:1 molar ratio, heat denatured and slowly cooled to allow the duplexed insert to form. The annealing of the oligos is such that single stranded DNA overhangs will be at each end, which will be compatible with Nhe I/EcoR I digested vector. Suitable oligos for use in this alternative method include:

| | |
|---|---|
| Cas.2-F2 | |
| 5′CT AGC tgt tga caa tta atc atc | (Seq. ID No. 10) |
| cgg ctc gta taa tgt gtg gaa ttg | |
| tga gcg gat aac aat tG 3′ | |
| Cas.2-B2 | |
| 5′ AAT TCa att gtt atc cgc tca caa | (Seq. ID No. 11) |
| ttc cac aca tta tac gag ccg gat | |
| gat taa ttg tca aca G 3′ | |

Following verification of the intermediate vector, a third cassette is inserted between the EcoR I and Kpn I sites of the modified f1. Cassette 3 (Seq. ID No. 12, see FIG. 4c) is the display cassette and contains an antibody cloning region and a recombinant gene III from pAX131-syn.gene III.

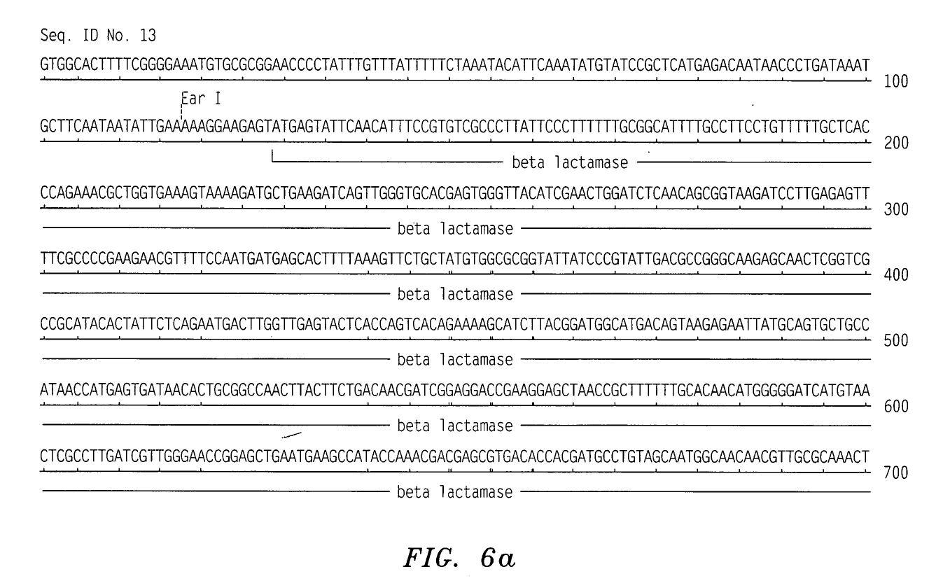

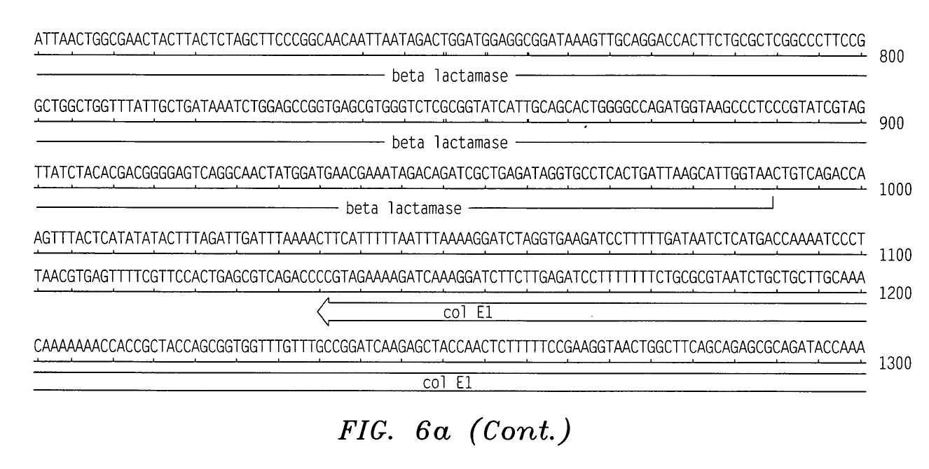

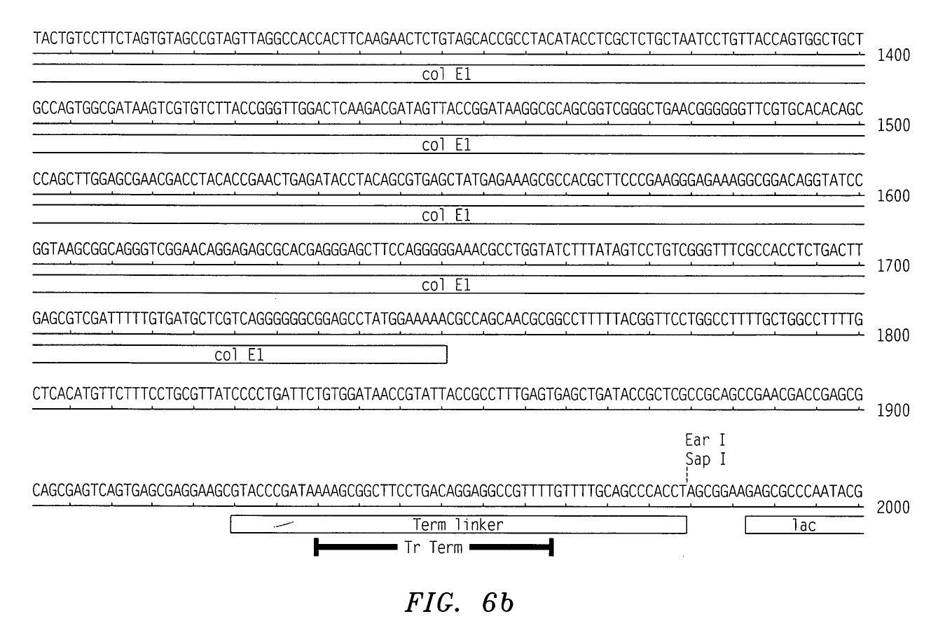

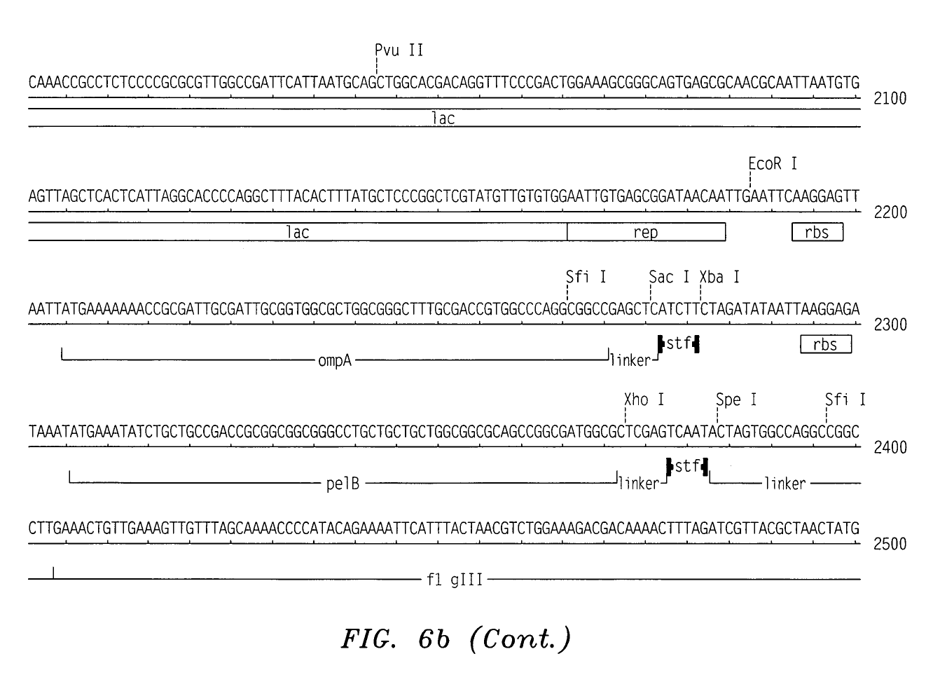

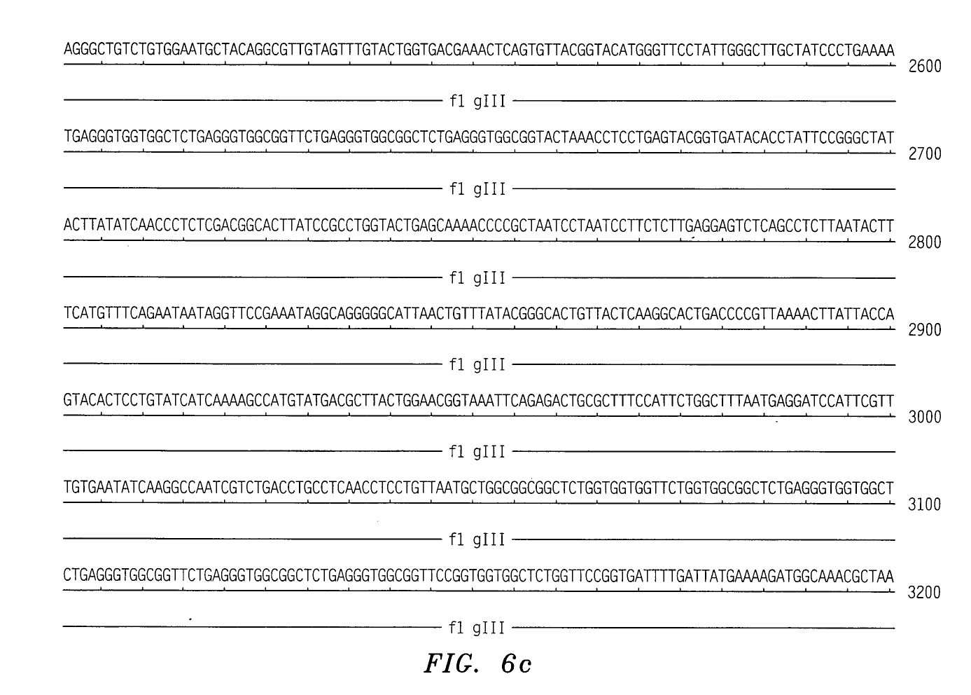

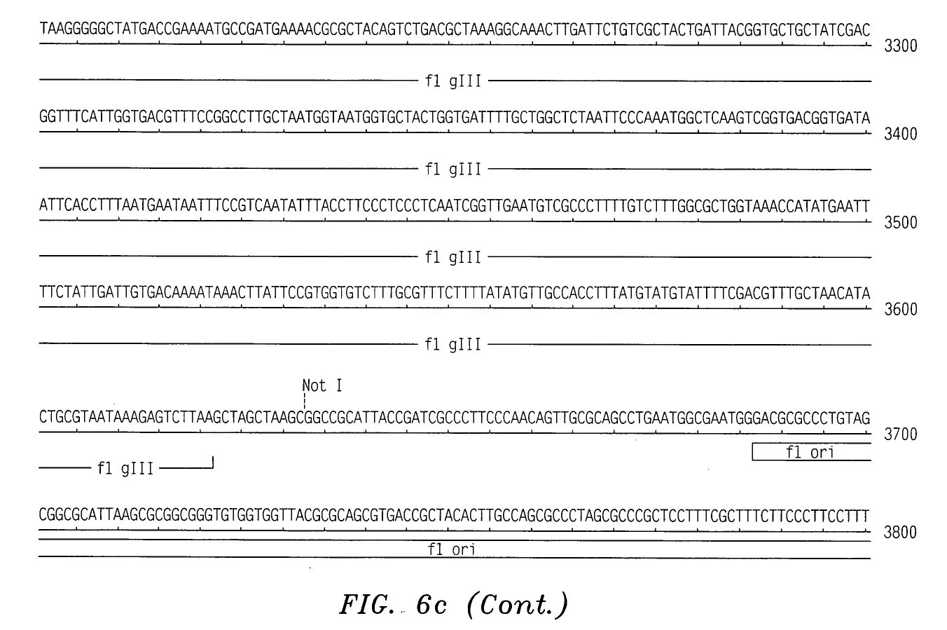

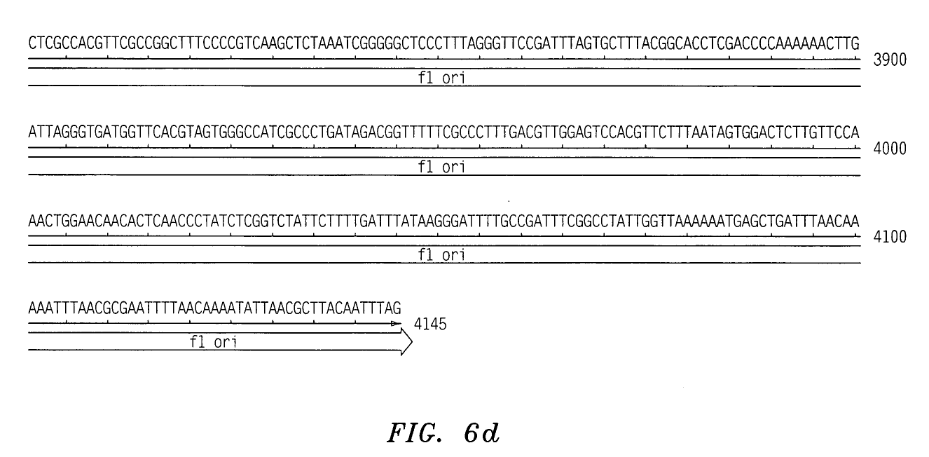

PAX131 is a phagemid vector prepared by modifying Bluescript II. FIG. 5 is a map of pAX131. FIGS. 6a-d show the nucleic acid sequence (Seq. ID No. 13) for pAX131. The preparation of pAX131 is described more fully in commonly owned pending application entitled PHAGEMID VECTORS filed on even date herewith under Express Mail Label No. EL820507456US (copy attached), the disclosure of which is incorporated herein in its entirety by this reference.

Preparation of Synthetic Gene III and the Display Cassette

The antibody cloning region of pAX131 was constructed using an overlapping oligo approach (synthetically generated region). The area of interest includes a ribosomal binding site followed by an optimized (for E. coli expression) ompA leader sequence, an Sfi I, then Sac I and Xba I cloning sites, another ribosomal binding sequence, an optimized pe1 B leader sequence, Xho I and Spe I cloning sites followed by a downstream Sfi I. The portion of pAX131 replaced includes the sequence for a gene III. See FIG. 5.

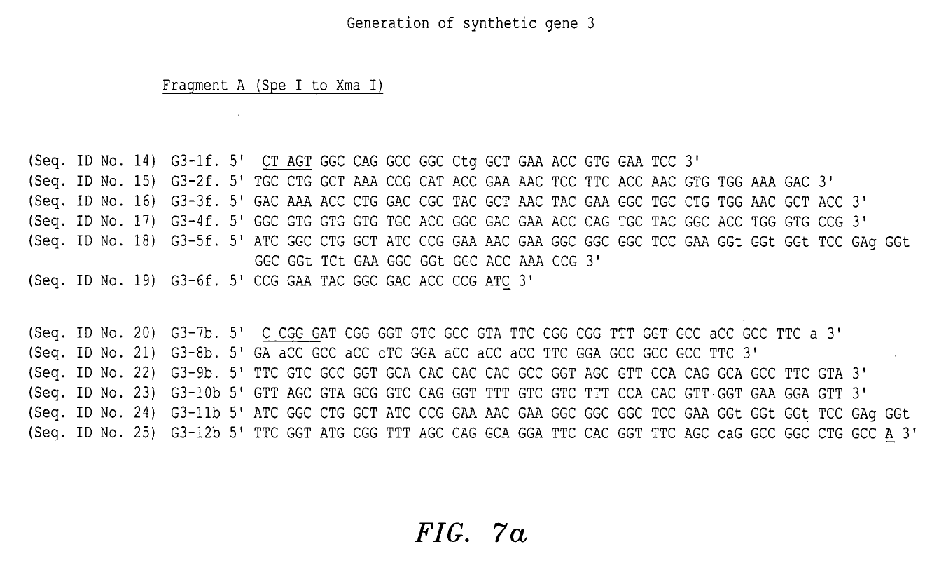

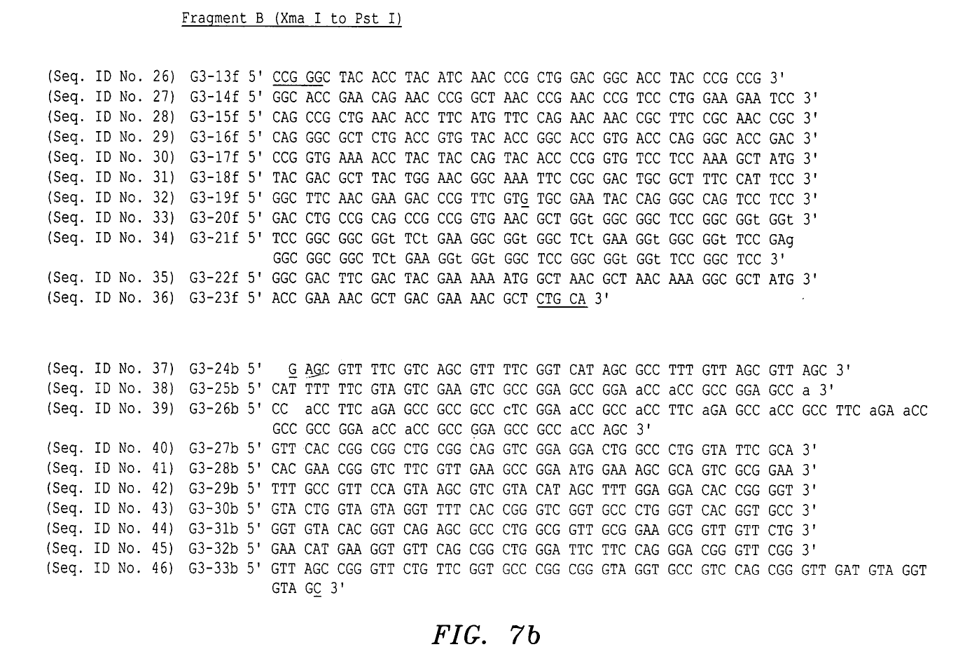

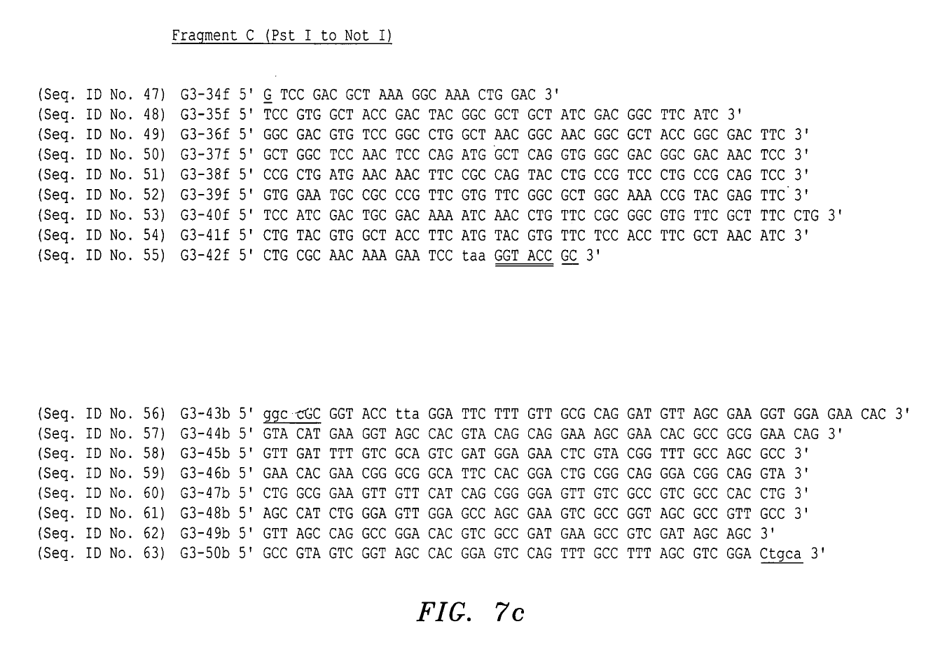

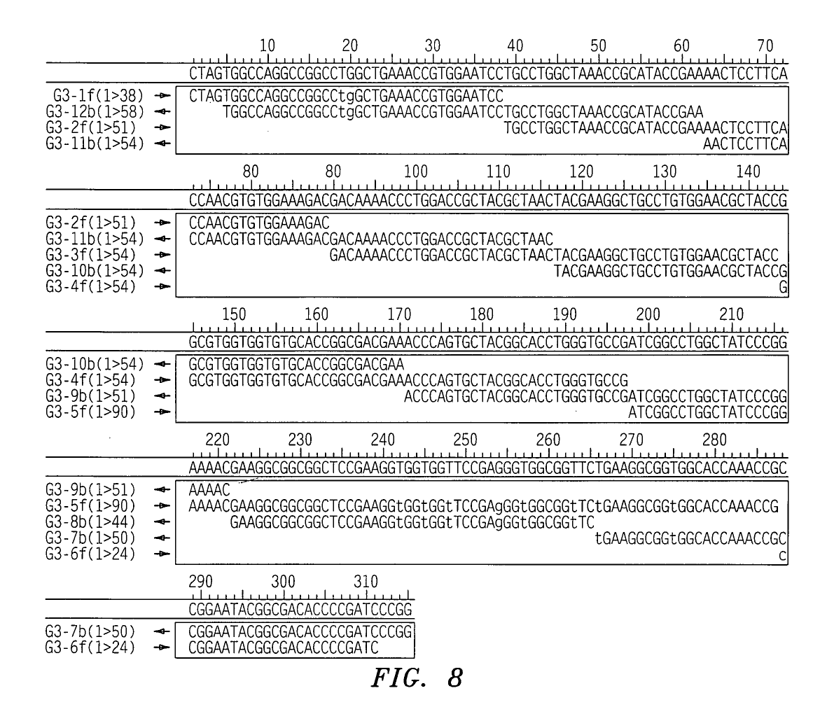

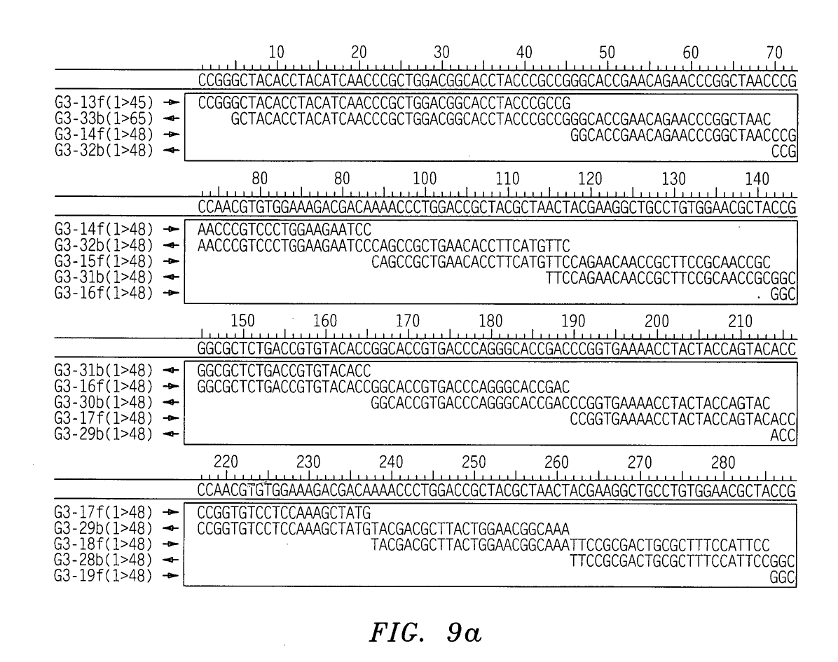

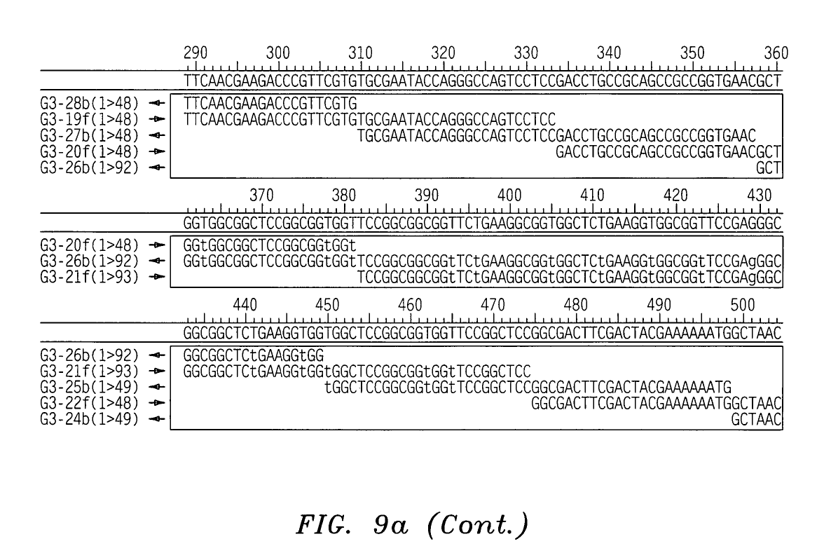

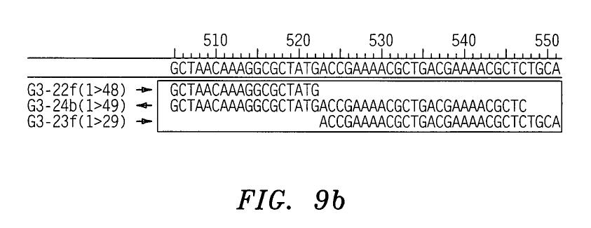

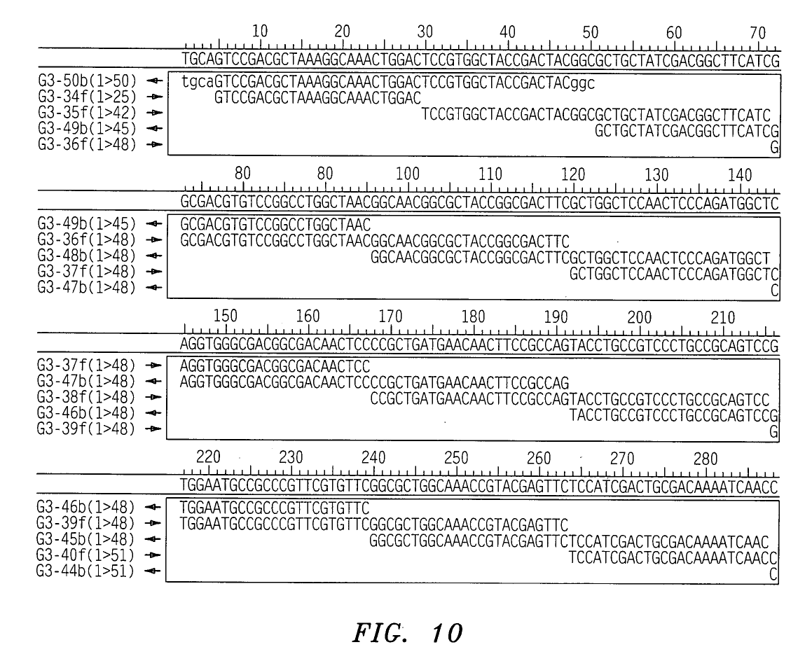

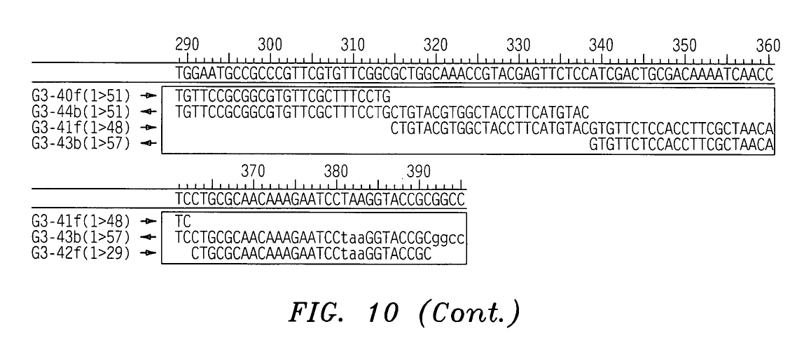

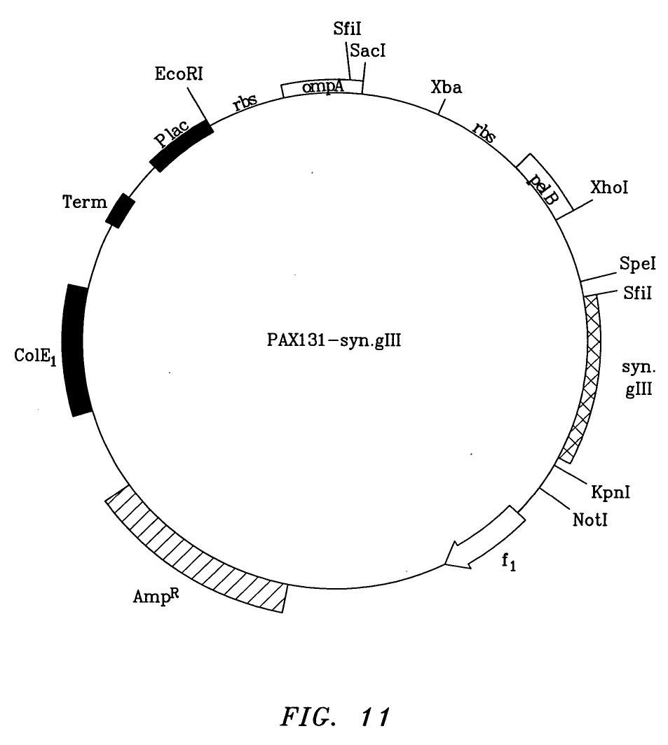

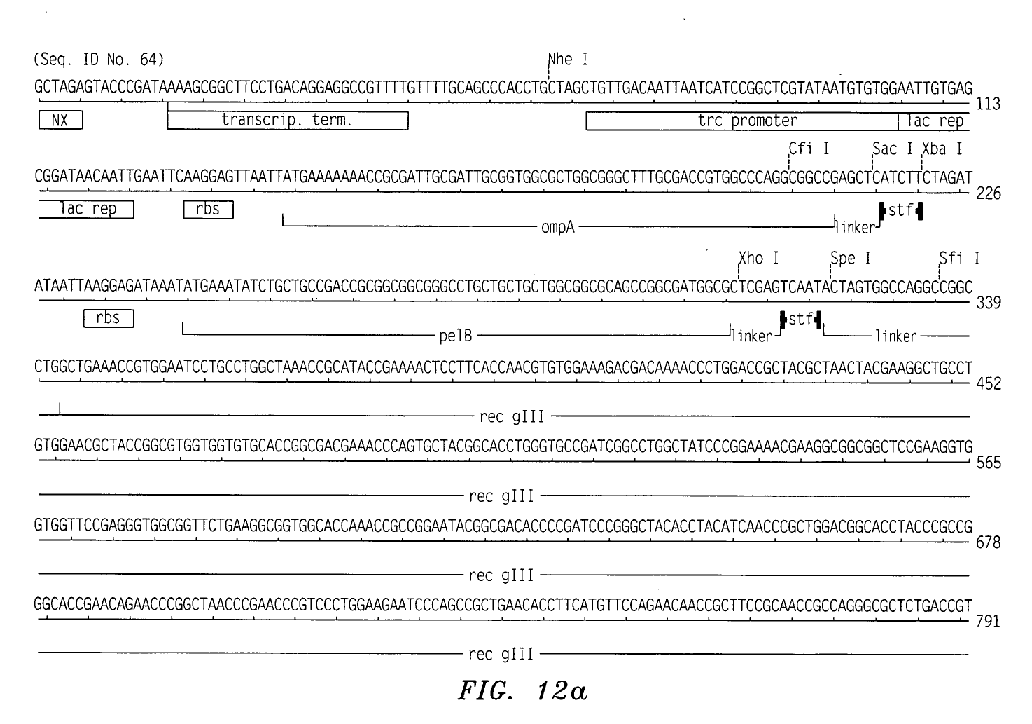

The nucleotide sequence of f1 gene III is optimized for bacterial expression. The gene is assembled using overlapping phosphorylated oligonucleotides ligated together and cloned initially as three fragments (A, B, and C) into pBluescript. However, in order to reduce the possibility of concatemerization of the insert on addition of ligase it may be desirable to not have 5′ phosphate modification on the oligos which form the 5′ ends of the fragments. Overlapping oligos for producing fragments A, B and C (Seq. ID Nos. 14-63) are given FIGS. 7a, 7b and 7c, respectively. The oligos are resuspended in sterile water and combined at equal molar ratios along with T4 DNA ligase buffer. Oligo sets corresponding to the individual fragments are combined and heat denatured. The oligos are then slowly cooled to room temperature allowing annealing to occur. FIGS. 8-10 show the alignment of the oligos for each of fragments A, B and C, respectively. All oligo sequences are shown in the sense orientation in these figures, meaning reverse oligos (designated with “b” at the end of the oligo name) are shown as their reverse complement in order to see the alignment with the forward oligos (designated with an “f” at the end of the oligo name). A portion of the annealed product is used in a ligation reaction with appropriately digested pBluescript using T4 DNA ligase and fresh ligase buffer. Individual clones of each fragment can be sequenced to identify the correct products. The correct sequence of fragment A is isolated as a Spe I/Xma I insert and ligated into the Spe I and Xma I digested vector containing fragment B. The combined fragment A+B is isolated by a Spe I to Pst I digest and combined as part of a three way ligation with the Pst I/Not I digested fragment C and Spe I/Not I digested pAX131 vector. The resulting vector pAX131-syn.gIII (see FIG. 11) is digested with Spe I and Kpn I to create the display cassette (cassette 3, see FIG. 4c) for insertion into the phage vector.

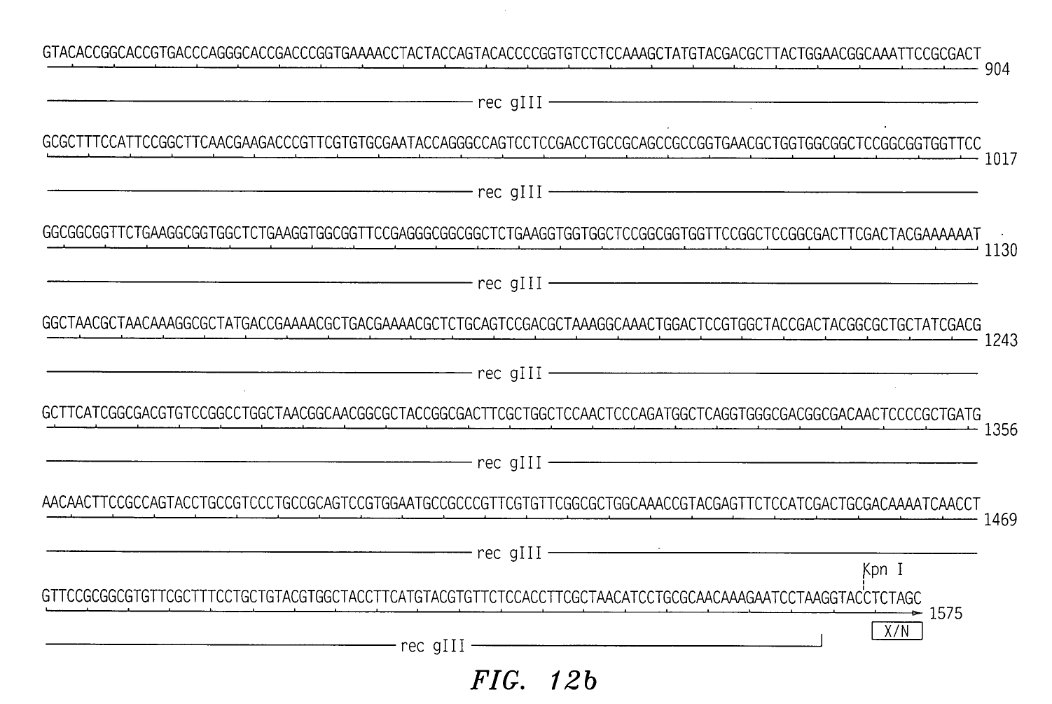

The sequence of the final inserted construct (Seq. ID. No. 64) resulting from the insertion of cassettes 1a, 2 and 3 is shown in FIG. 12. Verification of the final construct includes analysis of the resulting RF DNA and phage plaque size as described above. Additionally, a control antibody (such as antitetanus toxoid) is cloned into the phage using the Sac I/Xba I sites for the light chain and Xho I/Spe I sites for the heavy chain Fd. A test for proper expression of the Fab is performed by Western Blot analysis of phage particles using anti-Fab or anti-pIII reagents. A test panning experiment can also be performed to ensure that the Fab-fusion is presented on the phage surface and available for antigen selection. A phage mixture at a ratio of 1 specific phage/antibody into 10⁶ or more non-specific phage/antibody can be made and used as the starting sample. Following 3 to 4 rounds of panning, the specific antibody should have been selected and therefore present at a much higher ratio than the starting ratio. Solid phase panning can be performed by adding 10¹⁰-10¹² phage to an antigen coated microtiter well for 1-2 hours at 37°. Non-specific phage arc washed off with 0.5% Tween/PBS. Specific phage are eluted with low pH (such as 0.1M HCl, pH 2.2 with glycine) for 10 minutes at room temperature. Eluted phage are neutralized (with 2M Tris Base) and then added to bacterial cells to allow infection 15 minutes at room temperature. All cells/phage are plated in top agar on LB-agar plates and incubated overnight at 37°. The next day, phage are recovered from bacterial plaques by adding 5 mls media to each large petri dish and scrapping the top agar into 50 ml conical tubes. Agar debris is removed by centrifugation. Phage stock is used directly or concentrated by PEG precipitation if necessary (4% PEG 8000+0.5 M NaCl on ice for 30 minutes followed by centrifugation at 12,000×g for 20 minutes at 4°). The enriched phage can be reselected in additional rounds of panning, typically 3-4 rounds total.

### EXAMPLE 2

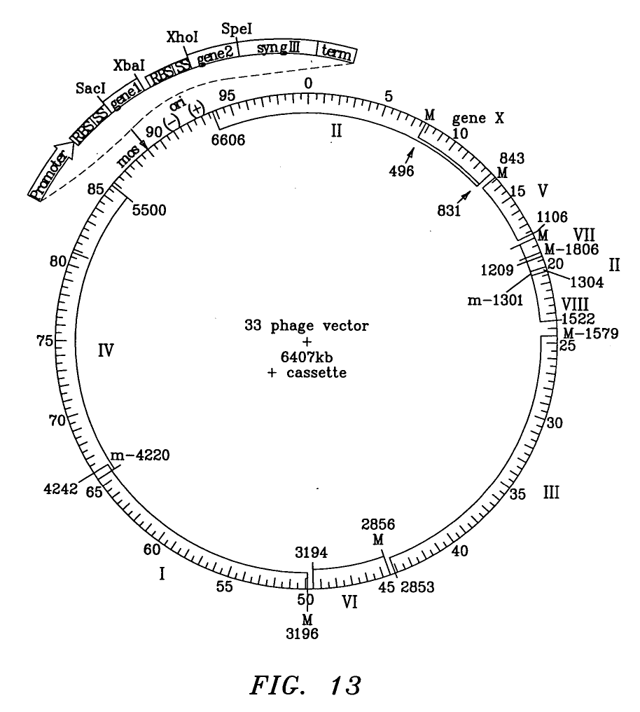

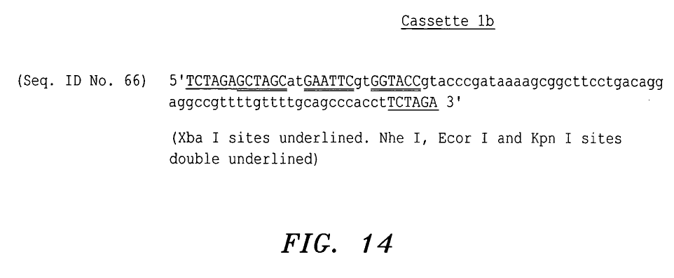

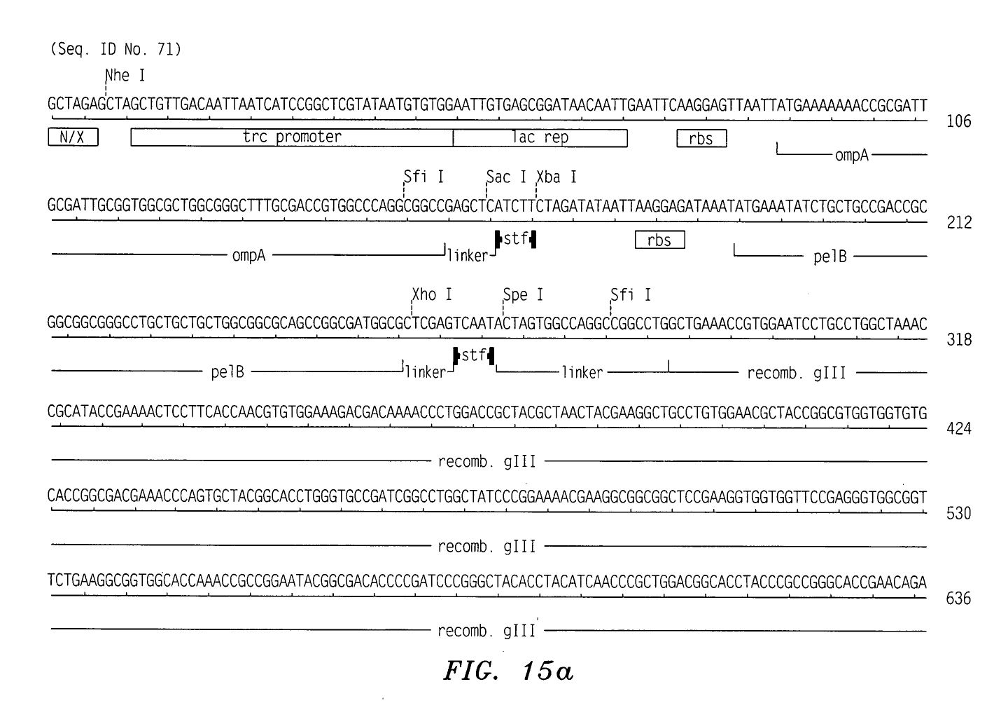

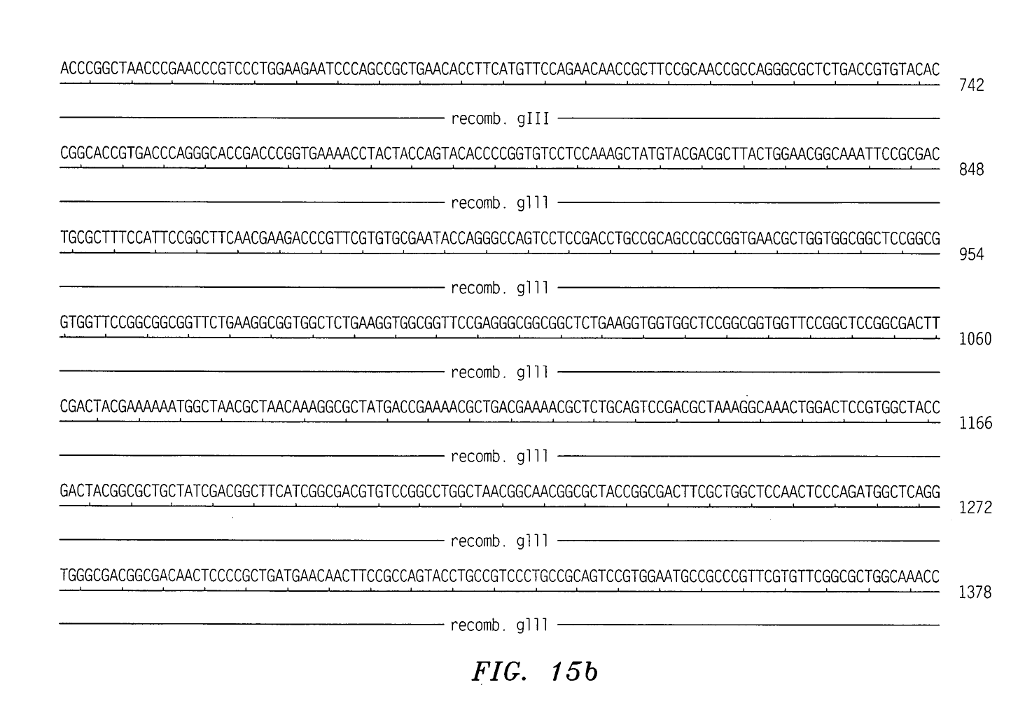

The overall scheme for modification of f1 at an alternate site is similar to that described above in Example 1. However, the insertion site for this Example is between the MOS hairpin and the minus origin. See FIG. 13. As above, the Nhe I site is incorporated using a mutagenic primer with f1 single stranded DNA template. The mutagenic primer used is 5′ GAA CGT GGC GAG AAA GCT AGC GAA GAA AGC GAA AGG 3′ (Seq. ID No. 65) where the double underlines indicate mutations introduced in order to create a Nhe I site (underlined). Cassette 1b (Seq. ID No. 66, see FIG. 14) is created by PCR using the following overlapping oligos: (Xba I sites underlined. Nhe I, EcoR I and Kpn I sites double underlined).

| | |
|---|---|
| Cas. 1b-F: | |
| 5′ gag gtt TCT AGA GCT AGC at GAA | (Seq. ID No. 67) |
| TTC gt GGT ACC gta ccc gat aaa agc | |
| ggc ttc ctg aca g 3′. | |
| Cas. 1b-B: | |
| 5′ aag cgt TCT AGA agg tgg gct gca | (Seq. ID No. 68) |
| aaa caa aac ggc ctc ctg tca gga | |
| agc cgc ttt tat c 3′. | |

An alternative method for creating Cassette 1b makes use of long complementary oligos which can form the double stranded DNA cassette. The two oligos are mixed together at a 1:1 molar ratio, heat denatured and slowly cooled to allow the duplexed insert to form. The annealing of the oligos is such that single stranded DNA overhangs will be at each end, such as would be created after an Xba I restriction digest. Suitable oligos for use in this alternative method include:

| | |
|---|---|
| Cas. 1b-F2 | |
| 5′ CT AGA GCT AGC at GAA TTC gt | (Seq. ID No. 69) |
| GGT ACC gta ccc gat aaa agc ggc | |
| ttc ctg aca gga ggc cgt ttt gtt | |
| ttg cag ccc acc tT 3′ | |
| Cas. 1b-B2 | |
| 5′ CTA GAa ggt ggg ctg caa aac aaa | (Seq. ID No. 70) |
| acg gcc tcc tgt cag gaa gcc gct | |
| ttt atc ggg tac GGT ACC ac GAA TTC | |
| at GCT AGC T 3′ | |

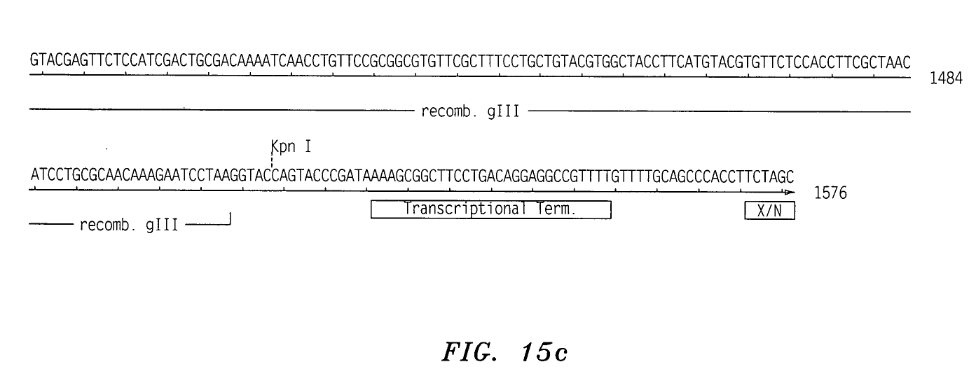

The remainder of the cloning strategy is identical to that described above in Example 1. The sequence of the final inserted construct (Seq. ID No. 71) resulting from the insertion of cassettes 1b, 2 and 3 is presented in FIG. 15. Likewise a test antibody is inserted into the second modified phage vector and analyzed for expression by Western Blot and by test panning.

It is contemplated that the present novel vector can be used in connection with the production and screening of libraries made in accordance with conventional phage display technologies. Both natural and synthetic antibody repertoires have been generated as phage displayed libraries. Natural antibodies can be cloned from B-cell mRNA isolated from peripheral blood lymphocytes, bone marrow, spleen, or other lymphatic tissue of a human or non-human donor. Donors with an immune response to the antigen(s) of interest can be used to create immune antibody libraries. Alternatively, non-immune libraries may be generated from donors by isolating naïve antibody B cell genes. PCR using antibody specific primers on the 1^{st} strand cDNA allows the isolation of light chain and heavy chain antibody fragments which can then be cloned into the display vector.

Synthetic antibodies or antibody libraries can be made up in part or entirely with regions of synthetically derived sequence. Library diversity can be engineered within variable regions, particularly within CDRs, through the use of degenerate oligonucleotides. For example, a single Fab gene may be modified at the heavy chain CDR3 position to contain random nucleotide sequences. The random sequence can be introduced into the heavy chain gene using an oligonucleotide which contains the degenerate coding region in an overlap PCR approach. Alternatively, degenerate oligo cassettes can be cloned into restriction sites that flank the CDR(s) to create diversity. The resulting library generated by such approaches can then be cloned into a display vector in accordance with this disclosure.

Upon introduction of the display library into bacteria, phage particles will be generated that have antibody displayed on the surface. The resulting collection of phage-displayed antibodies can be selected for those with the ability to bind to the antigen of interest using techniques known to those skilled in the art. Antibodies identified by this system can be used therapeutically, as diagnostic reagents, or as research tools.

It is contemplated that single and double stranded versions of the vectors described herein are within the scope of the present invention. It is well within the purview of those skilled in the art to prepare double stranded vectors from the single stranded nucleic acids described herein.

It will be understood that various modifications may be made to the embodiments described herein. For example, as those skilled in the art will appreciate, a first gene encoding an antibody light chain to be fused to and displayed by pIII and a second gene encoding a heavy chain Fd can be inserted into the vector at the newly created restriction site to provide effective antibody display. Therefore, the above description should not be construed as limiting, but merely as exemplifications of preferred embodiments. Those skilled in the art will envision other modifications within the scope and spirit of the claims appended hereto.

What is claimed:

1. A phage vector comprising:
a modified phage genome that contains, after gene IV but before the MOS hairpin:
a terminator;
a promoter,
a first cloning site adapted to receive a first gene encoding a polypeptide to be displayed; and
a nucleotide sequence encoding at least a functional domain of pIII.

2. A phage vector as in claim 1 wherein the polypeptide to be displayed includes a heavy chain Fd.

3. A phage vector as in claim 1 wherein the polypeptide to be displayed includes a light chain.

4. A phage vector as in claim 1 further comprising a second cloning site between the promoter and the first cloning site, the second cloning site being site adapted to receive a second gene encoding a polypeptide capable of dimerizing to the polypeptide to be displayed.

5. A phage vector as in claim 4 wherein the second gene encodes an antibody light chain.

6. A phage vector as in claim 1 wherein the second gene encodes an antibody heavy chain Fd.

7. A phage vector as in claim 1 wherein the nucleotide sequence encoding at least a functional domain of pIII encodes a truncated pIII.

8. A phage vector comprising
a modified phage genome that contains, after the MOS hairpin but before the minus strand origin:
a promoter;
a first cloning site adapted to receive a first gene encoding a polypeptide to be displayed;
a nucleotide sequence encoding at least a functional domain of pIII; and
a terminator.

9. A phage vector as in claim 8 wherein the polypeptide to be displayed includes a heavy chain Fd.

10. A phage vector as in claim 8 wherein the polypeptide to be displayed includes a light chain.

11. A phage vector as in claim 8 further comprising a second cloning site between the promoter and the first cloning site, the second cloning site being adapted to receive a second gene encoding a polypeptide capable of dimerizing to the polypeptide to be displayed.

12. A phage vector as in claim 11 wherein the second gene encodes an antibody light chain.

13. A phage vector as in claim 11 wherein the second gene encodes an antibody heavy chain Fd.

14. A phage vector as in claim 8 wherein the nucleotide sequence encoding at least a functional domain of pIII encodes a truncated pIII.

15. A method for producing a phage vector comprising:
incorporating a restriction site into a phage genome, the restriction site being located between gene IV and the MOS hairpin,
digesting at the incorporated restriction site; and
inserting a first cloning site for receiving a first gene encoding a polypeptide to be displayed and a nucleotide sequence encoding a pIII.

16. A method as in claim 15 wherein the first gene encodes an antibody heavy chain Fd.

17. A method as in claim 15 wherein the first gene encodes an antibody light chain.

18. A method as in claim 15 further comprising the step of inserting a second gene that encodes a polypeptide capable of dimerizing to said polypeptide to be displayed.

19. A method as in claim 18 wherein the second gene encodes an antibody light chain.

20. A method as in claim 18 wherein the second gene encodes an antibody heavy chain.

21. A method as in claim 15 wherein the nuclcotide sequence encoding at least a functional domain of pIII encodes a truncated pIII.

22. A method for producing a phage vector comprising:
incorporating a restriction site into a phage genome, the restriction site being located between the MOS hairpin and the minus strand origin;
digesting at the incorporated restriction site; and
inserting a first cloning site for receiving a first gene encoding a polypeptide to be displayed and a nucleotide sequence encoding at least a functional domain of pIII.

23. A method as in claim 22 wherein the first gene encodes an antibody heavy chain Fd.

24. A method as in claim 22 wherein the first gene encodes an antibody light chain.

25. A method as in claim 22 further comprising the step of inserting a second cloning site for receiving a second gene encoding a polypeptide capable of dimerizing to said polypeptide to be displayed.

26. A method as in claim 25 wherein the second gene encodes an antibody light chain.

27. A method as in claim 25 wherein the second gene encodes an antibody heavy chain.

28. A method as in claim 22 wherein the nucleotide sequence encoding at least a functional domain of pIII encodes a truncated pIII.

29. A phage display library produced using the vector of claim 1.

30. A phage display library produced using the vector of claim 8.

31. A vector produced by the method of claim 15.

32. A vector produced using the method of claim 22.

33. A phage vector comprising
a phage genome modified to contain a restriction site after gene IV but before the MOS hairpin.

34. A phage vector as in claim 33 wherein the restriction site is selected from the group consisting of Nhe I, Hind III, Nco I, Xma I, Bgl II, Bst I and Pvu I.

35. A phage vector as in claim 33 wherein the restriction site is an Nhe I site.

36. A phage vector comprising
a phage genome modified to contain a restriction site after the MOS hairpin but before the minus strand origin.

37. A phage vector as in claim 36 wherein the restriction site is selected from the group consisting of Nhe I, Hind III, Nco I, Xma I, Bgl II, Bst I and Pvu I.

38. A phage vector as in claim 36 wherein the restriction site is an Nhe I site.
